(19) United States
(12) Patent Application Publication
(10) Pub. No.: US 2017/0071836 A1
(43) Pub. Date: Mar. 16, 2017

(54) SULFATE-FREE COSMETIC CLEANSING AGENTS COMPRISING BIOSURFACTANTS

(71) Applicant: Henkel AG & Co. KGaA, Duesseldorf (DE)

(72) Inventors: Heike Schelges, Willich (DE); Maria Tretyakova, Dortmund (DE)

(73) Assignee: Henkel AG & Co. KGaA, Duesseldorf (DE)

(21) Appl. No.: 15/254,740

(22) Filed: Sep. 1, 2016

(30) Foreign Application Priority Data
Sep. 14, 2015 (DE) 10 2015 217 501.4

(51) Int. Cl. A61K 8/41; A61Q 19/10; A61Q 5/02; A61K 8/60; A61K 8/37

(52) U.S. Cl. CPC A61K 8/41; A61K 8/602; A61K 8/37; A61Q 5/02; A61Q 19/10; A61K 2800/596

(57) ABSTRACT

A sulfate surfactant-free cosmetic cleansing agent includes biosurfactants in combination with acyl lactylates as the surfactants. The cosmetic cleansing agent has an outstanding washing and foaming behavior.

### FIELD OF THE INVENTION

The present invention generally relates to a sulfate surfactant-free cosmetic cleansing agent.

### BACKGROUND OF THE INVENTION

Aqueous cosmetic cleansing agents usually include surfactant mixtures, wherein anionic, amphoteric, non-ionic and cationic surfactants may be present. Cleansing agents including these surfactants exhibit good cleansing power, and in particular a good foaming behavior. The majority of these surfactants, however, are entirely or partially obtained from petrochemicals. Yet, sustainability in the use of cosmetic ingredients is becoming increasingly important and something that a growing number of consumers and manufacturers of cosmetic cleansing agents is demanding.

Sulfate surfactants, such as sodium lauryl sulfate or sodium laureth sulfate, which exhibit an excellent foaming behavior, are frequently used surfactants in cosmetic cleansing agents. For a variety of reasons, however, consumers increasingly demand sulfate surfactant-free cosmetic cleansing agents. Sulfate surfactant-free cosmetic cleansing agents, however, generally have a poor foaming behavior.

Biosurfactants are surface-active substances of microbial origin, which can be produced based on vegetable oil or sugar substrates. These substrates may in part consist of agricultural waste such as rice husks or waste waters from the sugar industry, so that in this case also no starting materials for the production of foodstuffs are lost. Biosurfactants thus meet the requirements with regard to sustainability since they are produced from renewable resources. They are used both in household cleaners, detergents and dishwashing agents (for example, U.S. Pat. No. 5,520,839, DE 19600743 A1) and in a variety of cosmetic cleansing agents (for example, WO 2014/095367 A1, WO 2013/098066 A2).

WO 2014/095367 A1 discloses the use of biosurfactants in combination with anionic surfactants, such as sodium laureth sulfate. WO 2011/120776 A1 discloses biosurfactants in combination with mild anionic surfactants such as glycinates or sulfosuccinates, wherein a highly foam-boosting surfactant is preferably additionally present.

It is therefore desirable to provide sulfate surfactant-free cosmetic cleansing agents that, despite the absence of sulfate surfactants, have an excellent foaming behavior and washing behavior.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic cleansing agent according to the present invention includes biosurfactants in combination with acyl lactylates as the surfactants. A cosmetic cleansing agent having excellent foaming properties and cleansing properties may thus be provided, including ingredients, in particular the surfactants that are present, that are not very skin-irritating and easily biodegradable and can be obtained from renewable resources.

A cosmetic cleansing agent that, in each case based on the total weight of the cosmetic cleansing agent, includes: 1 to 50 wt. % biosurfactant(s), 0.5 to 30 wt. % of one or more C₁₀-C₂₀ acyl lactylates as an anionic surfactant, and less than 0.5 wt. % sulfate surfactants.

Use of a mixture of one or more biosurfactants and one or more C₁₀-C₂₀ acyl lactates as a surfactant component in cosmetic cleansing agents.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly, it was found that the above objectives can be achieved when the surfactants that the cosmetic cleansing agent includes are biosurfactants in combination with acyl lactylate surfactants, wherein in particular the excellent foaming behavior was not expected. At the same time, requirements with regard to sustainability and biodegradability of the ingredients included in the agent are met.

The present invention thus relates to a cosmetic cleansing agent that, in each case based on the total weight of the cosmetic cleansing agent, includes:

(a) 1 to 50 wt. % biosurfactant(s);
(b) 0.5 to 30 wt. % of one or more C₁₀-C₂₀ acyl lactylates as an anionic surfactant; and
(c) less than 0.5 wt. % sulfate surfactants.

The cosmetic cleansing agent according to the invention includes one or more biosurfactants as essential components.

Biosurfactants shall be understood to mean substances that are formed by microorganisms and often are also secreted from the cell. Like traditional surfactants, biosurfactants are surface-active substances that lower the surface tension of liquids and thereby promote the mixing of aqueous (hydrophilic) and water-repelling (hydrophobic) phases. Biosurfactants can be produced under mild production conditions that have a low need for energy consumption. In general, they are easily biodegradable, and the environmental compatibility thereof is very high. Moreover, they are not toxic, and no toxic by-products are generated during production of the same. Raw materials used for the microbial production of the same are carbohydrates, in particular sugar such as glucose, and/or lipophilic carbon sources such as fats, oils, partial glycerides, fatty acids, fatty alcohols, long-chain saturated or unsaturated hydrocarbons. According to the invention, the biosurfactants are preferably biosurfactants produced by way of fermentation.

The biosurfactants include glycolipids, lipopeptides, lipoproteins, fatty acids, phospholipids, neutral lipids and polymeric surfactants (such as emulsan), all of which may also be used in the present invention.

Glycolipids, which may be used in the present invention, are compounds in which one or more monosaccharide units are linked by glycosidic bonds to a lipid component. Examples of glycolipids as biosurfactants that can be used according to the invention are rhamnolipids, sophorolipids, mannosylerythritol lipids and trehalose lipids. Among these, rhamnolipids, sophorolipids, mannosylerythritol lipids and combinations thereof are preferred.

Rhamnolipids are produced by bacteria of the Pseudomonas genus, in particular Pseudomonas aeruginosa, preferably when grown on hydrophobic substrates such as n-alkanes or vegetable oils. Further glycolipids, such as glucose lipids, cellobiose lipids or trehalose lipids, are produced by different microorganisms on various substrates. Furthermore, mannosylerythritol lipids according to the invention are preferred glycolipid biosurfactants; these are produced by bacteria of the Pseudozyma sp. Candida antarctica and Ustilago sp.

Rhamnolipids according to the present invention have the following general formula:

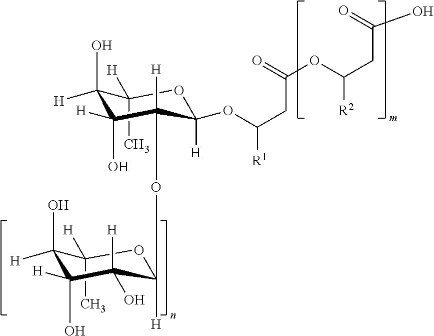

where m is 2, 1 or 0,
n is 1 or 0,
R¹ and R², independently of one another, are an identical or different organic group having 2 to 24, preferably 5 to 13 carbon atoms, in particular a substituted or unsubstituted, branched or unbranched alkyl group, which may also be unsaturated, wherein the alkyl group is preferably a linear saturated alkyl group having 8 to 12 carbon atoms, and more preferably a nonyl or decyl group or a mixture thereof.
Salts of these compounds are likewise covered by the present invention.

The term “di-rhamnolipid” in the present invention shall be understood to mean compounds of above formula or the salts thereof, in which n is 1.

Correspondingly, “mono-rhamnolipid” in the present invention shall be understood to mean compounds of the general formula or the salts thereof, in which n is 0.

According to the invention, mixtures of mono- and di-rhamnolipids can preferably be used. The ratio of mono-rhamnolipid to di-rhamnolipid is preferably approximately 2:1 to 4:1, and more preferably 2.5:1 to 3:1. Particularly preferred are mixtures of mono- and di-rhamnolipids in which R¹ and R² independently of one another represent a linear nonyl group or a decyl group in the above formula. In the latter case, this involves rhamnolipids that are each derived from 3-hydroxydodecanoic acid and/or 3-hydroxyundecanoic acid. Such mixtures are available commercially from Agae Technologies, USA, for example, under the designation Rhamnolipid R90, R95 or R98, wherein the number in each case indicates the purity level. Rhamnolipid R90 can be used particularly preferably according to the invention.

Sophorolipids are produced by way of fermentation using yeasts such as Candida bombicola (also known as Torulopsis bombicola), Yarrowia lipolytica, Candida apicola (Torulopsis apicola), and Candida bogoriensis by growing these on sugars, hydrocarbons, vegetable oils or mixtures thereof.

Sophorolipids have the below formulas (1) (lactone form) and (2) (free acid), wherein the two forms are usually present in a mixture,

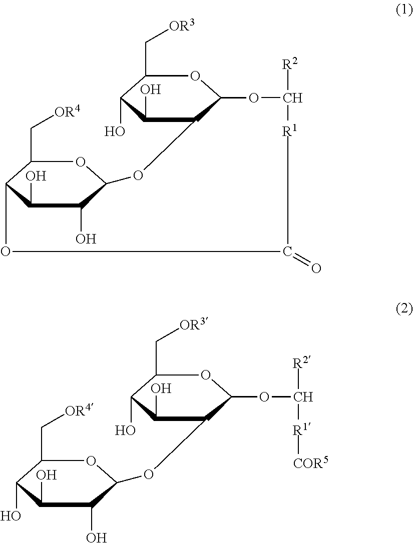

where R¹ and R^{1′}, independently of one another, represent saturated hydrocarbon chains or monounsaturated or polyunsaturated, in particular monounsaturated, hydrocarbon chains having 8 to 20, in particular 12 to 18 carbon atoms, and more preferably 14 to 18 carbon atoms, which may be linear or branched and may include one or more hydroxy groups, R² and R^{2′}, independently of one another, represent a hydrogen atom or a saturated alkyl group or a monounsaturated or polyunsaturated, in particular monounsaturated, alkyl group having 1 to 9 carbon atoms, and more preferably 1 to 4 carbon atoms, which may be linear or branched and may include one or more hydroxy groups, and R³, R^{3′}, R⁴ and R^{4′}, independently of one another, represent a hydrogen atom or an acetyl group.

Preferred sophorolipids are those in which R¹ and R^{1′} are a monounsaturated, linear hydrocarbon chain having 15 carbon atoms. It is furthermore preferred that R² and R^{2′} represent a methyl group or a hydrogen atom, and still more preferably each represents a methyl group.

According to the invention, preferred sophorolipids are those in which the acid form and the lactone form are present in a mixture, wherein preferably approximately 20 to approximately 60 wt. % of the sophorolipid is present in acid form and the remainder of the sophorolipid is present in lactone form.

In particular, preferred sophorolipids are those in which compounds of above formulas (1) and (2) are present in a mixture, where R¹ and R^{1′} are a monounsaturated, linear hydrocarbon chain having 14 to 18 carbon atoms, still more preferably 15 carbon atoms, R³ and R⁴ represent an acetyl group, R^{3′} and R^{4′} represent a hydrogen atom, and R² and R^{2′} represent a methyl group, and wherein approximately 20 to 60 wt. % of the sophorolipids is present in acid form.

Such sophorolipids are commercially available, for example from Soliance under the designation Sopholiance S. More precisely, the sophorolipid available from Soliance under the trade name Sopholiance S is an approximately 60 wt. % sophorolipid solution and is obtained, for example, by fermenting Candida bombicola on rapeseed oil methyl ester and glucose (INCI: Candida bombicola/glucose/methyl rapeseed ferment (and) Water). Sopholiance S is a preferred sophorolipid according to the invention.

In Soliance S, the free acid form accounts for approximately 20 wt. % in the mixture with the lactone form.

Mannosylerythritol lipids are glycolipids of the following general formula:

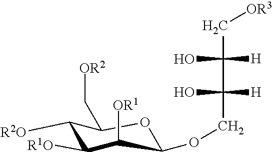

in which R¹ independently of one another represent fatty acid acyl groups having 4 to 24 carbon atoms, preferably 8 to 12 carbon atoms, R² independently of one another represent a hydrogen atom or an acetyl group, and R³ represents a hydrogen atom or a fatty acid acyl group having 2 to 24 carbon atoms. A suitable mannosylerythritol lipid according to the invention is commercially available under the designation Ceramela-B (Toyobo) (INCI: Pseudozyma Tsukubaensis/Olive Oil/Glycerin/Soy Protein Ferment).

The biosurfactants also include the substance group of lipids and lipid derivatives, which include in particular lipopeptides. In general, lipopeptides are non-ribosomally synthesized from the respective microorganisms, for example gram-positive bacteria, in particular of the Bacillus and Streptomyces genera, gram-negative bacteria, in particular of the Pseudomonas genus, and myxobacteria, and filamentous fungi. The peptide chains are normally composed of two to forty amino acids and may be linear, cyclic or branched. In contrast to ribosomally synthesized peptide chains, the monomeric building blocks they include are often not only proteinogenic L-amino acids, but also D-amino acids and carboxylic acids and/or alpha-hydroxycarboxylic acids of all kinds. The amino acids are usually L-α- or D-α-amino acids; however, it is also possible for β-, γ- or δ-amino acids to be present, which may likewise be present in a D or else in an L configuration. The peptide chains can also include further chemical modifications; in particular, they may be glycosylated, hydrolyzed, N-methylated or N-formylated. Furthermore, thiazoline and/or oxazoline rings in various oxidation states are frequently occurring structural elements. Surfactin is a well-known lipopeptide biosurfactant, which has the following structure and is generally used as an alkali or ammonium salt:

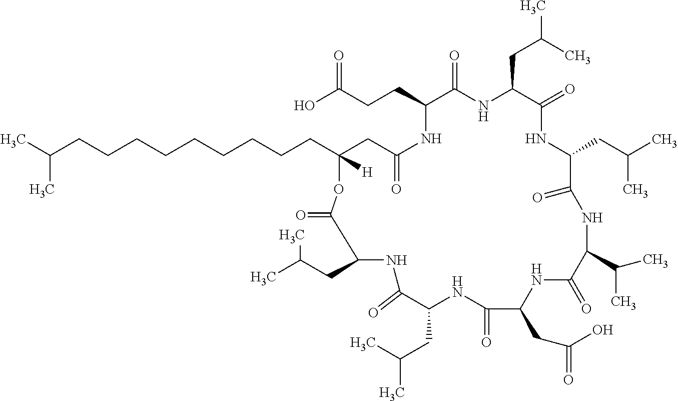 [SMILES: CC(C)CCCCCCCCC[C@@H]1CC(=O)N[C@@H](CCC(=O)O)C(=O)N[C@@H](CC(C)C)C(=O)N[C@H](CC(C)C)C(=O)N[C@@H](C(C)C)C(=O)N[C@@H](CC(=O)O)C(=O)N[C@H](CC(C)C)C(=O)N[C@@H](CC(C)C)C(=O)O1]

A surfactin that is suitable according to the invention is commercially available from Kaneka.

The lipopeptides that may preferably be used as biosurfactants according to the invention furthermore include those known as fatty acyl glutamates. These have the following general formula:

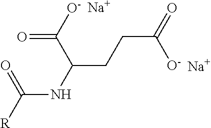

where R is a straight or branched alkyl chain having 5 to 21 carbon atoms, preferably 7 to 17 carbon atoms, and more preferably 12 to 16 or 13 to 15 carbon atoms. Fatty acyl glutamates, used as biosurfactants, are usually present in a mixture in which R has different chain lengths. The group R may also be hydroxylated, in particular monohydroxylated, wherein in this case a hydroxylation at the β-position is preferred. Fatty acyl glutamates, used as biosurfactants, are available from Modular Genetics, Inc., USA, for example.

The lipopeptides that may preferably be used as biosurfactants according to the invention furthermore include those known as fatty acyl glycinates. These have the following general formula:

RC(O)NHCH₂CO₂X,

where
R is a straight or branched alkyl chain having 5 to 21 carbon atoms, preferably 7 to 17 carbon atoms, and more preferably 12 to 16 or 13 to 15 carbon atoms, and
X is a cation, preferably an alkali metal or ammonium cation, more preferably a sodium or ammonium cation, or —H.
   Fatty acyl glycinates, used as biosurfactants, can also present in a mixture in which R can have different chain lengths.
   Fatty acyl glycinates, used as biosurfactants, are available from Modular Genetics, Inc., USA, for example.

According to the invention, preferred cosmetic cleansing agents are those that include the following biosurfactants or biosurfactant combinations: rhamnolipid(s), sophorolipid(s), fatty acyl glutamate, fatty acyl glycinates, fatty acyl glutamate and mannosylerythritol lipid(s).

The cosmetic cleansing agent includes the biosurfactants in an amount of approximately 1 to 50 wt. %, preferably approximately 1 to 25 wt. %, more preferably approximately 2 to 15 wt. %, and more preferably 3 to 10 wt. %, based on the total weight of the cleansing agent. If biosurfactant mixtures are present, the percentage information refers to the total amount of biosurfactants present.

The cosmetic composition according to the invention includes one or more C₁₀-C₂₀ acyl lactates as a further essential component. Acyl lactates are known anionic surfactants, which today can be obtained according to the relevant methods of preparative organic chemistry. A method for producing the same includes, for example, allowing fatty acid and lactic acid to react in the presence of NaOH or KOH at 140° C. to 210° C. for a total of 3 to 10 hours, and to continuously remove the condensation water from the equilibrium. The lactylate thus produced, having a free acid content, can be neutralized as needed using various alkaline compounds or amines.

According to the invention, the acyl lactylates have the following formula,

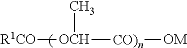

in which R¹CO denotes a linear or branched acyl group having 10 to 20 carbon atoms, n denotes numbers from 1 to 3, M denotes hydrogen, an alkali metal and/or alkaline earth metal, ammonium, alkyl ammonium, alkanol ammonium or glucammonium. In preferred embodiments of the invention, n denotes 2. Examples of acyl lactylate that may be used according to the invention are lactylates derived from fatty acids having 10 to 20 carbon atoms, such as capric acid, lauric acid, isotridecanoic acid, myristic acid, palmitic acid, isopalmitic acid, palmoleic acid, stearic acid, isostearic acid, oleic acid, elaidic acid, petroselinic acid, linoleic acid, linolenic acid, eleostearic acid, arachyinic acid and gadoleic acid, combinations thereof, and the technical mixtures thereof, which develop, for example, in the hydrolysis of natural fats and oils under pressure or the reduction of aldehydes from Roelen's oxo synthesis. Preferred lactylates according to the invention are those derived from fatty acids having 12 to 18 carbon atoms, such as lauric acid, isotridecanoic acid, myristic acid, palmitic acid, isopalmitic acid, palmoleic acid, stearic acid, isostearic acid, oleic acid, elaidic acid, petroselinic acid, linoleic acid, linolenic acid and eleostearic acid. More preferred are stearoyl lactate, isostearoyl lactate, palmitoyl lactylate and/or lauroyl lactylate. According to the invention, lauroyl lactylate is particularly preferred, and in particular sodium lauroyl-2-lactylate. Sodium lauroyl-2-lactylate is commercially available, for example as a 30 wt. % solution under the name Hydriol LLA (Hydrior, Switzerland).

The cosmetic cleansing agent includes the acyl lactylates in an amount of approximately 0.5 to 30 wt. %, preferably approximately 1 to 20 wt. %, more preferably approximately 1.5 to 15 wt. %, and more preferably 3 to 10 wt. %, based on the total weight of the cleansing agent. If acyl lactylate mixtures are present, the percentage information refers to the total amount of acyl lactylates present.

One essential aspect of the present invention is that the cosmetic cleansing agent includes 0.5 wt. % or less of sulfate surfactants. It is more preferred if the cosmetic agent according to the invention includes less than 0.2 wt. % sulfate surfactants, more preferably less than 0.1 wt. %, and still more preferably no sulfate surfactants. According to the invention, sulfate surfactants shall be understood to mean those surfactants that include a sulfate group as the ionic group. Examples are lauryl sulfates and lauryl ether sulfates.

In preferred embodiments of the invention, the cosmetic cleansing agent does not include any further surfactant or at least less than 0.5 wt. % further surfactant, preferably less than 0.2 wt. %, and still more preferably less than 0.1 wt. % further surfactant, in addition to the biosurfactant and the acyl lactylate.

According to the invention, the cosmetic cleansing agent includes water as the cosmetic carrier. Other customary carriers such as ethanol and glycol may be present in embodiments of the invention, but it is preferred with respect to the biodegradability and natural availability of the raw materials if the cosmetic cleansing agent according to the invention only includes water as the carrier.

According to the invention, the cosmetic cleansing agent may include further customary ingredients of cosmetic cleansing agents. Examples of such customary ingredients are aromatic substances or perfumes, preservatives, nourishing agents and/or thickeners. The present invention, however, is not limited to these additional ingredients.

Preservatives that may be present are preferably organic acids or salts thereof, such as phenoxyethanol, methylparaben, ethylparaben, sodium benzoate, sodium salicylate and combinations thereof. Due to the antibacterial action of the surfactants, however, it is also possible according to the invention to formulate cosmetic agents that are free of preservatives. If a preservative is present, this is preferably present in an amount of 0.05 to 5 wt. %, preferably 0.1 to 3 wt. %, more preferably 0.2 to 2 wt. %, and likewise preferably 0.5 to 1.0 wt. %, based on the total weight of the cleansing agent in each case. If multiple preservatives are present, the percentage information refers to the total amount of preservatives.

According to the invention, in particular natural aromatic substances are preferred as aromatic substances or perfumes that may be present in the cosmetic cleansing agent. If an aromatic substance is present, this is preferably present in an amount of 0.05 to 2 wt. %, preferably 0.1 to 1.5 wt. %, more preferably 0.2 to 1 wt. %, and likewise preferably 0.5 to 1 wt. %, based on the total weight of the cleansing agent in each case. If multiple aromatic substances are present, the percentage information refers to the total amount of aromatic substances or perfumes.

The agent can include oleosomes as nourishing agents, for example, preferably natural oleosomes such as vegetable oils, plant extracts, but also monosaccharides or oligosaccharides and/or lipids. Aloe vera extracts or olive oil shall be mentioned as examples. Glycerol is another suitable nourishing agent, which also acts as a humectant. If a nourishing agent is present, this is preferably present in an amount of 0.1 to 15 wt. %, preferably 0.5 to 10 wt. %, more preferably 1 to 7.5 wt. %, based on the total weight of the cleansing agent in each case. If multiple nourishing agents are present, the percentage information refers to the total amount of nourishing agents.

Preferred thickeners according to the invention are thickeners of plant origin, such as polysaccharides and celluloses (cellulose itself and the derivatives thereof), alginic acid (and the corresponding physiologically safe salts thereof, the alginates), agar agar (including the polysaccharide agarose present as the main component in agar agar), starch fractions and derivatives such as amylose, amylopectin and dextrins, karaya gum, gellan gum, locust bean gum, gum arabic, dextrans, guar gum and xanthan gum, or combinations thereof. Guar gum, gellan gum and/or xanthan gum are preferred thickeners for the present invention. Synthetic thickeners such as polyacrylates are preferably not present.

Suitable cellulose derivatives include methyl celluloses, ethyl celluloses, hydroxyalkyl celluloses (such as hydroxyethyl cellulose), methyl hydroxyalkyl celluloses and carboxymethyl celluloses (INCI: Cellulose Gum) and the physiologically safe salts thereof.

If a thickener is present, this is preferably present in an amount of 0.01 to 30 wt. %, preferably 0.05 to 20 wt. %, more preferably 0.1 to 10 wt. %, and likewise preferably 0.5 to 5 wt. %, based on the total weight of the cosmetic cleansing agent in each case. If multiple thickeners are present, the percentage information refers to the total amount of thickeners.

According to the invention, the cosmetic cleansing agent may be present as a shampoo, shower gel, liquid hand soap, facial cleansing agent, facial gel or other known cosmetic formulation forms.

The present invention furthermore relates to the use of a mixture of one or more biosurfactants and one or more C₁₀-C₂₀ acyl lactates as a surfactant component in cosmetic cleansing agents. What was said above for the cosmetic agent applies, mutatis mutandis, to preferred embodiments of the use.

### Table Overview:

Preferred cosmetic cleansing agents for the cleansing agent system according to the invention are listed hereafter. The information is provided in % by weight in each case and refers to the active substance concentration.

| | Formula | Formula | Formula | Formula |
|---|---|---|---|---|
| | 1a | 2a | 3a | 4a |
| Biosurfactant | 1 to 50 | 1 to 25 | 2 to 15 | 3 to 10 |
| C₁₀-C₂₀ acyl lactylate | 0.5 to 30 | 1 to 20 | 1.5 to 15 | 2 to 10 |
| Sulfate surfactants | ≦0.5 | ≦0.5 | ≦0.5 | ≦0.5 |
| Misc | to make up | to make up | to make up | to make up |
| | to 100 | to 100 | to 100 | to 100 |
| | Formula | Formula | Formula | Formula |
| | 1b | 2b | 3b | 4b |
| Biosurfactant | 1 to 50 | 1 to 25 | 2 to 15 | 3 to 10 |
| C₁₀-C₂₀ acyl lactylate | 0.5 to 30 | 1 to 20 | 1.5 to 15 | 2 to 10 |
| Thickener | 0.01 to 30 | 0.05 to 20 | 0.1 to 10 | 0.5 to 5 |
| Sulfate surfactants | ≦0.5 | ≦0.5 | ≦0.5 | ≦0.5 |
| Misc | to make up | to make up | to make up | to make up |
| | to 100 | to 100 | to 100 | to 100 |
| | Formula | Formula | Formula | Formula |
| | 5a | 6a | 7a | 8a |
| Rhamnolipid | 1 to 50 | 1 to 25 | 2 to 15 | 3 to 10 |
| C₁₂-C₁₈ acyl lactylate | 0.5 to 30 | 1 to 20 | 1.5 to 15 | 2 to 10 |
| Sulfate surfactants | ≦0.1 | ≦0.1 | ≦0.1 | ≦0.1 |
| Misc | to make up | to make up | to make up | to make up |
| | to 100 | to 100 | to 100 | to 100 |
| | Formula | Formula | Formula | Formula |
| | 5b | 6b | 7b | 8b |
| Rhamnolipid | 1 to 50 | 1 to 25 | 2 to 15 | 3 to 10 |
| C₁₂-C₁₈ acyl lactylate | 0.5 to 30 | 1 to 20 | 1.5 to 15 | 2 to 10 |
| Guar gum, gellan gum | 0.01 to 30 | 0.05 to 20 | 0.1 to 10 | 0.5 to 5 |
| and/or xanthan gum | | | | |
| Sulfate surfactants | ≦0.1 | ≦0.1 | ≦0.1 | ≦0.1 |
| Misc | to make up | to make up | to make up | to make up |
| | to 100 | to 100 | to 100 | to 100 |
| | Formula | Formula | Formula | Formula |
| | 5c | 6c | 7c | 8c |
| Mixture of mono- and di- | 1 to 50 | 1 to 25 | 2 to 15 | 3 to 10 |
| rhamnolipids that are each | | | | |
| derived from 3- | | | | |
| hydroxydodecanoic acid | | | | |
| and/or 3- | | | | |
| hydroxyundecanoic acid | | | | |
| Lauroyl-2-lactylate, in | 0.5 to 30 | 1 to 20 | 1.5 to 15 | 2 to 10 |
| particular Na-lauroyl-2- | | | | |
| lactylate | | | | |
| Guar gum, gellan gum | 0.01 to 30 | 0.05 to 20 | 0.1 to 10 | 0.5 to 5 |
| and/or xanthan gum | | | | |
| Sulfate surfactants | None | none | None | none |
| Misc | to make up | to make up | to make up | to make up |
| | to 100 | to 100 | to 100 | to 100 |
| | Formula | Formula | Formula | Formula |
| | 9a | 10a | 11a | 12a |
| Sophorolipid | 1 to 50 | 1 to 25 | 2 to 15 | 3 to 10 |
| C₁₂-C₁₈ acyl lactylate | 0.5 to 30 | 1 to 20 | 1.5 to 15 | 2 to 10 |
| Sulfate surfactants | ≦0.1 | ≦0.1 | ≦0.1 | ≦0.1 |
| Misc | to make up | to make | to make up | to make up |
| | to 100 | up to 100 | to 100 | to 100 |
| | Formula | Formula | Formula | Formula |
| | 9b | 10b | 11b | 12b |
| Sophorolipid, 20 to 60 wt. | 1 to 50 | 1 to 25 | 2 to 15 | 3 to 10 |
| % in acid form | | | | |
| C₁₂-C₁₈ acyl lactylate | 0.5 to 30 | 1 to 20 | 1.5 to 15 | 2 to 10 |
| Guar gum, gellan gum | 0.01 to 30 | 0.05 to 20 | 0.1 to 10 | 0.5 to 5 |
| and/or xanthan gum | | | | |
| Sulfate surfactants | ≦0.1 | ≦0.1 | ≦0.1 | ≦0.1 |
| Misc | to make up | to make | to make up | to make up |
| | to 100 | up to 100 | to 100 | to 100 |
| | Formula | Formula | Formula | Formula |
| | 9c | 10c | 11c | 12c |
| Sopholiance S | 1 to 50 | 1 to 25 | 2 to 15 | 3 to 10 |
| Lauroyl-2-lactylate, in | 0.5 to 30 | 1 to 20 | 1.5 to 15 | 2 to 10 |
| particular Na-lauroyl-2- | | | | |
| lactylate | | | | |
| Guar gum, gellan gum | 0.01 to 30 | 0.05 to 20 | 0.1 to 10 | 0.5 to 5 |
| and/or xanthan gum | | | | |
| Sulfate surfactants | None | none | None | none |
| Misc | to make up | to make | to make up | to make up |
| | to 100 | up to 100 | to 100 | to 100 |
| | Formula | Formula | Formula | Formula |
| | 13a | 14a | 15a | 16a |
| Fatty acyl glutamate | 1 to 50 | 1 to 25 | 2 to 15 | 3 to 10 |
| C₁₂-C₁₈ acyl lactylate | 0.5 to 30 | 1 to 20 | 1.5 to 15 | 2 to 10 |
| Sulfate surfactants | ≦0.1 | ≦0.1 | ≦0.1 | ≦0.1 |
| Misc | to make | to make up | to make up | to make up |
| | up to 100 | to 100 | to 100 | to 100 |
| | Formula | Formula | Formula | Formula |
| | 13b | 14b | 15b | 16b |
| Fatty acyl glutamate of C₈-C₁₈ | 1 to 50 | 1 to 25 | 2 to 15 | 3 to 10 |
| fatty acid | | | | |
| C₁₂-C₁₈ acyl lactylate | 0.5 to 30 | 1 to 20 | 1.5 to 15 | 2 to 10 |
| Guar gum, gellan gum | 0.01 to 30 | 0.05 to 20 | 0.1 to 10 | 0.5 to 5 |
| and/or xanthan gum | | | | |
| Sulfate surfactants | ≦0.1 | ≦0.1 | ≦0.1 | ≦0.1 |
| Misc | to make | to make up | to make up | to make up |
| | up to 100 | to 100 | to 100 | to 100 |
| | Formula | Formula | Formula | Formula |
| | 13c | 14c | 15c | 16c |
| Fatty acyl glutamate of | 1 to 50 | 1 to 25 | 2 to 15 | 3 to 10 |
| C₁₄-C₁₆ fatty acid | | | | |
| Lauroyl-2-lactylate, in | 0.5 to 30 | 1 to 20 | 1.5 to 15 | 2 to 10 |
| particular Na-lauroyl-2- | | | | |
| lactylate | | | | |
| Guar gum, gellan gum | 0.01 to 30 | 0.05 to 20 | 0.1 to 10 | 0.5 to 5 |
| and/or xanthan gum | | | | |
| Sulfate surfactants | None | none | none | none |
| Misc | to make | to make up | to make up | to make up |
| | up to 100 | to 100 | to 100 | to 100 |
| | Formula | Formula | Formula | Formula |
| | 17a | 18a | 19a | 20a |
| Fatty acyl glutamate and | 1 to 50 | 1 to 25 | 2 to 15 | 3 to 10 |
| mannosylerythritol lipid | | | | |
| C₁₂-C₁₈ acyl lactylate | 0.5 to 30 | 1 to 20 | 1.5 to 15 | 2 to 10 |
| Sulfate surfactants | ≦0.1 | ≦0.1 | ≦0.1 | ≦0.1 |
| Misc | to make | to make up | to make up | to make up |
| | up to 100 | to 100 | to 100 | to 100 |
| | Formula | Formula | Formula | Formula |
| | 17b | 18b | 19b | 20b |
| Fatty acyl glutamate of C₈-C₁₈ | 1 to 50 | 1 to 25 | 2 to 15 | 3 to 10 |
| fatty acid and | | | | |
| mannosylerythritol lipid | | | | |
| C₁₂-C₁₈ acyl lactylate | 0.5 to 30 | 1 to 20 | 1.5 to 15 | 2 to 10 |
| Guar gum, gellan gum | 0.01 to 30 | 0.05 to 20 | 0.1 to 10 | 0.5 to 5 |
| and/or xanthan gum | | | | |
| Sulfate surfactants | ≦0.1 | ≦0.1 | ≦0.1 | ≦0.1 |
| Misc | to make | to make up | to make up | to make up |
| | up to 100 | to 100 | to 100 | to 100 |
| | Formula | Formula | Formula | Formula |
| | 17c | 18c | 19c | 20c |
| Fatty acyl glutamate of | 1 to 50 | 1 to 25 | 2 to 15 | 3 to 10 |
| C₁₄-C₁₆ fatty acid and | | | | |
| mannosylerythritol lipid | | | | |
| Lauroyl-2-lactylate, in | 0.5 to 30 | 1 to 20 | 1.5 to 15 | 2 to 10 |
| particular Na-lauroyl-2- | | | | |
| lactylate | | | | |
| Guar gum, gellan gum | 0.01 to 30 | 0.05 to 20 | 0.1 to 10 | 0.5 to 5 |
| and/or xanthan gum | | | | |
| Sulfate surfactants | None | none | none | none |
| Misc | to make | to make up | to make up | to make up |
| | up to 100 | to 100 | to 100 | to 100 |
| | Formula | Formula | Formula | Formula |
| | 21a | 21a | 21a | 21a |
| Fatty acyl glycinate | 1 to 50 | 1 to 25 | 2 to 15 | 3 to 10 |
| C₁₂-C₁₈ acyl lactylate | 0.5 to 30 | 1 to 20 | 1.5 to 15 | 2 to 10 |
| Sulfate surfactants | ≦0.1 | ≦0.1 | ≦0.1 | ≦0.1 |
| Misc | to make | to make up | to make up | to make up |
| | up to 100 | to 100 | to 100 | to 100 |
| | Formula | Formula | Formula | Formula |
| | 21b | 21b | 21b | 21b |
| Fatty acyl glycinate of C₈-C₁₈ | 1 to 50 | 1 to 25 | 2 to 15 | 3 to 10 |
| fatty acid | | | | |
| C₁₂-C₁₈ acyl lactylate | 0.5 to 30 | 1 to 20 | 1.5 to 15 | 2 to 10 |
| Guar gum, gellan gum | 0.01 to 30 | 0.05 to 20 | 0.1 to 10 | 0.5 to 5 |
| and/or xanthan gum | | | | |
| Sulfate surfactants | ≦0.1 | ≦0.1 | ≦0.1 | ≦0.1 |
| Misc | to make | to make up | to make up | to make up |
| | up to 100 | to 100 | to 100 | to 100 |
| | Formula | Formula | Formula | Formula |
| | 21c | 21c | 21c | 21c |
| Fatty acyl glycinate of C₁₂-C₁₆ | 1 to 50 | 1 to 25 | 2 to 15 | 3 to 10 |
| fatty acid | | | | |
| Lauroyl-2-lactylate, in | 0.5 to 30 | 1 to 20 | 1.5 to 15 | 2 to 10 |
| particular Na-lauroyl-2- | | | | |
| lactylate | | | | |
| Guar gum, gellan gum | 0.01 to 30 | 0.05 to 20 | 0.1 to 10 | 0.5 to 5 |
| and/or xanthan gum | | | | |
| Sulfate surfactants | None | none | none | none |
| Misc | to make | to make up | to make up | to make up |
| | up to 100 | to 100 | to 100 | to 100 |

“Misc” according to the invention shall essentially be understood to mean water, if necessary in combination with another cosmetic carrier; the cosmetic carrier, however, preferably only includes water. “Misc” may optionally also cover further customary ingredients of cosmetic cleansing agents such as preservatives, pH regulators such as acids, and/or aromatic substances.

### Examples

The following cosmetic cleansing agents shown in the tables were produced. The percentage information shall be understood to mean percent by weight, in each case based on the total weight of the cleansing agent.

TABLE 1

| INCI or other | | Exam- | Exam- | Exam- | Exam- |
|---|---|---|---|---|---|
| designation | Example 1 | ple 2 | ple 3 | ple 4 | ple 5 |
| Rhamnolipid R-90 | 5.55 | — | — | — | — |
| Sophorolipid - | — | — | 8.66 | — | — |
| Sopholiance S | | | | | |
| Fatty acyl glutamate | — | — | — | 5.00 | 5.00 |
| Fatty acyl glycinate | — | 5.00 | — | — | — |
| Mannosylerythritol | — | — | — | — | 0.50 |
| lipid | | | | | |
| Hydriol LLA 30% | 16.67 | 16.67 | 16.67 | 16.67 | 16.67 |
| Sodium lauroyl-2- | | | | | |
| lactylate | | | | | |
| Glycerol, 99.5% | 7.00 | 7.00 | 1.00 | — | — |
| Perfume | 0.70 | 0.70 | 1.00 | 0.50 | 0.50 |
| Citric acid | 2.61 | 2.61 | 2.61 | — | |
| monohydrate | | | | | |
| regular | | | | | |
| Keltrol SF | 0.80 | 0.80 | 0.80 | — | — |
| Xanthan gum | | | | | |
| Guar gum | — | — | — | 0.90 | 0.90 |
| Phenonip ® ME - | — | — | 1.00 | — | — |
| Phenoxyethanol, | | | | | |
| methylparaben, | | | | | |
| ethylparaben | | | | | |
| Phenoxyethanol | — | — | — | 0.8 | 0.80 |
| Sodium salicylate | — | — | — | — | 0.40 |
| Sodium benzoate | — | — | — | — | 0.23 |
| Water, demineralized | up to 100 | up | up | up | up |
| | | to 100 | to 100 | to 100 | to 100 |

All exemplary compositions were set to a pH of 7.

In all the examples, an outstanding overall cosmetic performance, including washing behavior and sensation on the skin/the hair, was observed. In particular, the foaming behavior was excellent in all cases where the product was applied and was comparable to the foaming behavior of a combination of biosurfactants with SLES. Due to the antibacterial action of the biosurfactants, it was also possible to provide compositions without preservatives (Example 1).

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic cleansing agent that, in each case based on the total weight of the cosmetic cleansing agent, includes:
(a) 1 to 50 wt. % biosurfactant(s);
(b) 0.5 to 30 wt. % of one or more C₁₀-C₂₀ acyl lactylates as an anionic surfactant; and
(c) less than 0.5 wt. % sulfate surfactants.

2. The cosmetic cleansing agent according to claim 1, including
(a) 2 to 10 wt. % of the biosurfactant (a) and
(b) 1 to 20 wt. % of the acyl lactylate (b),
(c) each based on the total weight of the cosmetic cleansing agent.

3. The cosmetic cleansing agent according to claim 1, including a glycolipid, a lipopeptide or a combination thereof as the biosurfactant.

4. The cosmetic cleansing agent according to claim 1, including a rhamnolipid, a sophorolipid, a mannosylerythritol, a surfactin, a fatty acyl glutamate, a fatty acyl glycinate or a combination thereof as the biosurfactant.

5. The cosmetic cleansing agent according to claim 4, including a sophorolipid as the biosurfactant, wherein 20 to 60 wt. % is present in the acid form.

6. The cleansing agent according to claim 4, including a mixture of mono- and di-rhamnolipids that are each derived from 3-hydroxydodecanoic acid and/or 3-hydroxyundecanoic acid as the biosurfactant.

7. The cosmetic cleansing agent according to claim 1, wherein the acyl lactylate is selected from one or more C₁₂-C₁₈ acyl lactylates.

8. The cosmetic cleansing agent according to claim 1, wherein the acyl lactylate is selected from the group consisting of stearoyl lactate, isostearoyl lactate, palmitoyl lactylate and lauroyl lactylate.

9. The cosmetic cleansing agent according to claim 1, wherein the acyl lactylate is a lauroyl lactylate.

10. The cosmetic cleansing agent according to claim 1, including less than 0.1 wt. % sulfate surfactants.

11. The cosmetic cleansing agent according to claim 1, including less than 0.5 wt. % further surfactant other than the surfactants (a) and (b).

12. The cosmetic cleansing agent according to claim 1, further including a thickener, a preservative, an aromatic substance or a perfume, a nourishing agent or a combination thereof.

13. The cosmetic cleansing agent according to claim 1, which is formulated as a shampoo or a shower gel.
